(19) United States
(12) Patent Application Publication
(10) Pub. No.: US 2026/0294309 A1
(43) Pub. Date: Oct. 1, 2026

(54) MEDICAL PROBE BUFFER FOR BUFFERING A MEASURED PHYSIOLOGICAL SIGNAL

(71) Applicant: Sencure BV, Roden (NL)

(72) Inventors: Egbert BOUWMEESTER, Markelo (NL); Benjamin DE JONGE, Enschede (NL); Jelle Ferdinand TIMMERMANS, Borne (NL)

(73) Assignee: Sencure BV, Roden (NL)

(21) Appl. No.: 19/475,627

(22) PCT Filed: Apr. 3, 2024
(86) PCT No.: PCT/NL2024/050166

§ 371 Date: Oct. 15, 2025

(30) Foreign Application Priority Data
Apr. 17, 2023 (NL) 2034596

(51) Int. Cl. A61B 5/276

(52) U.S. Cl. CPC A61B 5/276; A61B 2562/182

(57) ABSTRACT

The invention relates to a medical probe buffer for buffering a measured physiological signal of a human body, comprising: at least one receive port couplable to a receive wire, which is arranged for conducting a signal based on a physiological signal of the human body; each receive port having an associated receive buffer arranged for buffering the signal from the respective receive port, wherein each receive buffer comprises an input, and an output; each receive port having an associated receive resistor conductively arranged between the respective receive port and the input of the respective receive buffer; at least one transmit port couplable to a transmit wire, which is arranged for conducting a signal to the human body; each transmit port having an associated transmit buffer arranged for buffering the signal provided to the respective transmit port, wherein each transmit buffer comprises an input, and an output; and each transmit port having an associated transmit resistor conductively arranged between the output of the transmit buffer and the transmit port; wherein a resistor group is formed by the receive resistor and the transmit resistor; and wherein the resistance values of the resistors in the resistor group are preselected such that a current through the human body does not exceed a safety threshold current when an electrical short-circuit fault occurs in a resistor in the resistor group.

### FIELD OF THE INVENTION

The invention relates to a medical probe buffer for buffering a measured physiological signal of a human body. The invention further relates to a medical probe and medical device incorporating the medical probe buffer.

### BACKGROUND OF THE INVENTION

A subset of medical electrical equipment measures one or more physiological signals of a human body with the use of electrical signals. This subset of medical electrical equipment, while measuring a physiological signal, should comply with the safety requirements specified in the IEC 60601 standard, more specific the IEC 60601-1 standard, even more specific the most recent version being the IEC 60601-1 version 3.1 standard.

One of the requirements in the IEC 60601-1 standard requires the medical electrical equipment to be tolerant and/or safe when one or multiple fault(s), for example short circuits, occurs in electrical components of the medical electrical equipment. A common solution is to arrange one or more resistors in series in each electrical wire. The resistors should have a resistance high enough to limit the current through the human body from which a physiological signal is measured to a current below a safety threshold current as specified in the IEC 60601-1 standard. Three series resistors may be preselected to strike a balance between lowering resistor values and limiting the number of additional electrical components.

The resistance value of the series resistors is typically such high that these three series resistors attenuate the electrical signal in that wire significantly. Furthermore, each of the three series resistors is a noise source decreasing the signal to noise ratio —SNR— of the electrical signal in that wire. The significant attenuation of the electrical signal as well as the decrease in SNR each individually and certainly in combination prohibits accurate measurement of physiological signals when complying to the IEC 60601-1 standard.

### SUMMARY OF THE INVENTION

An object of the invention is to overcome one or more of the disadvantages mentioned above. According to a first aspect of the invention, a medical probe buffer for buffering a measured physiological signal of a human body, comprising: at least one receive port couplable to a receive wire, which is arranged for conducting a signal based on a physiological signal of the human body; each receive port having an associated receive buffer arranged for buffering the signal from the respective receive port, wherein each receive buffer comprises an input, and an output; each receive port having an associated receive resistor conductively arranged between the respective receive port and the input of the respective receive buffer; at least one transmit port couplable to a transmit wire, which is arranged for conducting a signal to the human body; each transmit port having an associated transmit buffer arranged for buffering the signal provided to the respective transmit port, wherein each transmit buffer comprises an input, and an output; and each transmit port having an associated transmit resistor conductively arranged between the output of the transmit buffer and the transmit port; wherein a resistor group is formed by the receive resistor and the transmit resistor; and wherein the resistance values of the resistors in the resistor group are preselected such that a current through the human body does not exceed a safety threshold current when an electrical short-circuit fault occurs in a resistor in the resistor group.

The human body generates or carries physiological signals measurable by a medical probe. The physiological signal may be a bioelectrical signal, or an electrical signal associated with the human body. The human body generates or carries signals such as brain or nerve activity. The human body may also have electrical measurable properties, such as electrical skin resistance. The human body may also have electrical measurable characteristics, such as translucency measured by an electrically powered LED and an electrically powered light sensitive sensor. In all these examples electrical current may be exchanged between, or brought in close proximity of or contact with the human body and the medical device measuring, receiving, generating, providing and/or exchanging this electricity with the use of the medical device. Patient safety is safeguarded by the IEC 60601-1 standard. One of the requirements in the standard is that the patient safety is guaranteed in situations where one fault, more specific one short circuit, occurs in electrical components in the medical device which are part of the means of patient protection, abbreviated as MOP or MOPP. As one MOP may fail, such as a short circuit in a resistor being part of the MOP, medical devices are required to have double MOP. A further requirement according to the IEC 60601-1 standard is that the medical device should remain safe for the patient when apart from one failure in the double MOP, the rest of the medical device outside the MOP entirely fails, such as short circuits.

The medical device typically comprises a medical probe comprising a medical probe buffer for providing an electrical buffer between the medical device and the human body. The buffer typically adapts the impedances of the signals between the rest of the medical device and the human body. The buffer typically also amplifies the signals from the human body to the rest of the medical device. The buffer further has the function to ensure that the medical device complies to the IEC 60601-1 standard.

A voltage is defined as an electrical potential difference between two points. A reference electrical potential may be provided as a reference from which the electrical difference can be measured, thus measuring the voltage. The reference electrical potential is transmitted by the medical probe buffer to the human body, and may be typically labelled ground, electrical ground, or electrical patient ground.

Conductively coupled should be interpreted as that an electrically conductive path is present between the components conductively coupled. The electrically conductive path may comprise a conductor directly conductively connecting the components. Alternatively, the electrically conductive path may comprise next to a conductor other passive or even active electrical elements.

A resistor is typically a physical and typically a discrete component having a predefined resistance to electricity. The resistor typically has two ports typically having no orientation preference. In the context of the safety requirements of the IEC 60601-1 standard the resistor is typically arranged as a discrete component at some distance on for example a PCB as component only having tracks on the PCB leading to and from the two ports of the resistor. The distance and/or separation material between the tracks on the PCB ensure the resilience to short-circuits between the tracks. A port of the medical probe buffer is typically a connection point for connecting the medical probe buffer with the medical probe.

The group of resistors typically comprises all resistors that are conductively arranged between a port couplable to a probe cable comprising a wire and preferable a shield, and a buffer. The buffers typically comprise at least one output and at least one input. Depending on the direction of the signal, the at least one output or the at least one input of the buffer is conductively connected with a port.

The group of electrical components may be defined as all the components of the medical probe buffer conducting electricity. Typically, the group of electrical components comprises all the discrete electrical components. In some embodiments of the invention, the different buffers may be arranged in one IC, whereby the IC replaces the buffers integrated in the IC in the group of electrical components. The safety should still be guaranteed when an electrical short-circuit occurs in one of the electrical components part of one of the MOPs. The group of resistors is a subset of the electrical components and form the MOPs, but is arranged such that this group of resistors, when one electrical short-circuit occurs, still allows the medical probe buffer to comply to the requirement of the current through the human body not exceeding the threshold safety current.

The safety threshold current is specified in the IEC 60601-1 standard. The safety threshold current is depending on the use of the medical probe. For example, different requirements apply for invasive medical probes compared to non-invasive medical probes. The preselection of the resistance value of the resistor is typically based on the voltage divided by the safety threshold current applicable for the medical device, more specific for the medical probe. Preselection in the context of the current application is to be understood as determining upfront or before use of the medical probe buffer a resistance value of the resistor, and permanently setting or fixating this resistance value during the use. Preselection is typically done by selecting a particular resistor having a fixed resistor value and placing this resistor value permanently in the medical probe buffer.

Resistors are attenuating signals measured. Furthermore, resistors are noise sources decreasing the SNR of the measured signal. Arranging in the reception or transmission path only one resistor, such as the safety resistor mentioned for patient safety, provides an improved conductive path between the human body and the rest of the medical device. Further, to ensure the patient safety, two resistors, such as safety resistors for patient safety, although degrading the conductivity of the transitive path between the transmit buffer and the human body, the influence of these two resistors on the at least one received signal by the at least one received port is reduced. Hence, rearranging the resistors according to the invention provides the technical effect of improving the SNR and/or reducing the attenuation of the received signal based on the physiological signal of the human body.

According to another aspect, a medical probe comprises: a medical probe buffer according to any of the mentioned embodiments; and at least one probe cable comprising: a receive wire couplable to the at least one receive port; preferably a receive wire shield associated with the receive wire and couplable to the at least one receive shield port associated with the at least one receive port; and a transmit wire couplable to the at least one transmit port. The medical probe provides similar or even the same advantages as the medical probe buffer.

According to another aspect, a medical device for measuring a physiological signal of a human body, comprising: a medical probe arranged for measuring the physiological signal; a medical probe buffer according to any of the embodiments according to the invention, arranged for receiving the measured physiological signal from the probe; and a processor for processing the buffered physiological signal from the medical probe buffer. The medical device provides similar or even the same advantages as the medical probe buffer.

### DETAILED DESCRIPTION OF EXEMPLARY EMBODIMENTS

In an embodiment of the medical probe buffer, the number of receive ports is at least two. Comprising at least two receive ports implies that the medical probe buffer also comprises at least two receive resistors each associated with a receive port, and at least two receive buffers each also associated with a receive port. This embodiment improves the technical effect as typically at least one resistor less is present in the medical probe buffer eliminating a noise source as well as taking an attenuating element out of the conductive path of the received signal. Hence, rearranging the resistors according to the invention provides the enhanced technical effect of improving the SNR and/or reducing the attenuation of each of the at least two received signal based on the respective physiological signals of the human body.

In an embodiment of the medical probe buffer, the transmit resistor comprises a number of transmit resistors connected in series; and the transmit resistor in the resistor group is replaced with the number of transmit resistors. The function of the resistors in the resistor group is to ensure that the current through the patient does not exceed the safety threshold current even when an electrical short-circuit failure occurs, more specific an electrical short-circuit failure occurs bridging one of the resistors in the resistor group. As safety is to be ensured when an electrical short-circuit bridges a resistor, the remaining resistors should provide enough resistance such that the current through the human body or patient does not exceed the safety threshold current. The safety resistance is defined as the lowest resistance of the resistor group during an electrical short-circuit bridging randomly one resistor of the resistor group. During normal operation, the resistor group as a whole is providing the accumulated resistance, which is the accumulation of the resistances of the resistors in the resistor group forming a conductive path. The accumulated resistance is higher than the safety resistance. The accumulated resistance therefore reduces the SNR and/or increases the attenuation of the received signal based on the physiological signal of the human body. This reduction and/or increase is reduced by increasing the number of resistors in the resistor group. Increasing the number of resistors in the resistor group, wherein the resistors maintain the safety resistance, reduces the accumulated resistance. Thus, increasing the number of resistors in the resistor group advantageously improves the SNR and/or reduces the attenuation of the received signal based on the physiological signal of the human body.

In a further embodiment of the medical probe buffer, the number of transmit resistors connected in series is two. Increasing the number of resistors has the disadvantage of introducing more components that may fail, thereby decreasing the mean time between failure. A balance between increasing the number of resistors and limiting the number of resistors is advantageously found in preselecting two transmit resistors.

In an embodiment of the medical probe buffer, the medical probe comprises: a receive shield port associated with one of the at least one receive port and couplable to a shield arranged for electrically shielding the receive wire coupled to the associated receive port; each receive shield port having an associated shield buffer comprising an input and an output; and each receive shield port having an associated receive shield resistor conductively arranged between the output of the receive shield buffer and the receive shield port; wherein the resistor group is extended with the receive shield resistor. Connecting the shielding of the coupled receive wire comprising a shield advantageously increases the SNR. The resistor group is advantageously extended with the receive shield resistor asserting the current through the human body cannot exceed the safety threshold current during an electrical short-circuit fault.

Connecting the shielding of the coupled wire comprising a shield with a shield port advantageously increases the SNR. The resistor group is advantageously extended with the shield resistor asserting the current through the human body cannot exceed the safety threshold current during an electrical short-circuit fault. The shield port may be associated with a transmit and/or receive wire.

In a further embodiment of the medical probe buffer, the receive shield resistor comprises a number of receive shield resistors connected in series; and the receive shield resistor in the resistor group is replaced with the number of receive shield resistors. Increasing the number of resistors in the resistor group advantageously improves the SNR and/or reduces the attenuation of the received signal based on the physiological signal of the human body, as previously discussed.

In a further embodiment of the medical probe buffer, the number of receive shield resistors in series is two. Increasing the number of resistors in the resistor group advantageously improves the SNR and/or reduces the attenuation of the received signal based on the physiological signal of the human body, as previously discussed.

In a further embodiment of the medical probe buffer, the at least one receive shield buffer comprises a receive shield amplifier comprising a receive shield amplifier output, a receive shield amplifier negative input, and a receive shield amplifier positive input; the receive shield amplifier output is conductively coupled to the output of the receive shield buffer; the receive shield amplifier positive input is conductively coupled to the receive shield buffer input; the medical probe buffer comprises a receive shield feedback resistor conductively coupling the associated receive shield port and the receive shield amplifier negative input; and the resistor group is extended with the receive shield feedback resistor. The shield advantageously limits the electrical signature of the shielded wire, such as the susceptibility of the wire for external interference. Feeding the received signal back to the shield of the wire conducting the received signal advantageously reduces the resistance, preferably the impedance, of the shielded wire such that it becomes transparent or invisible, or almost transparent or invisible for the receive buffer input. Limiting the electrical signature also reduces the susceptibility to external signals such as external noise sources irradiating onto the shielded wire. This reduced susceptibility advantageously improves the SNR.

In an embodiment of the medical probe buffer, the at least one transmit buffer comprises a transmit amplifier comprising a transmit amplifier output, a transmit amplifier negative input, and a transmit amplifier positive input; the transmit amplifier output is conductively coupled to the output of the transmit buffer; the transmit amplifier positive input is conductively coupled to the transmit buffer input; the medical probe buffer comprises a transmit feedback resistor conductively coupling the associated transmit port and the transmit amplifier negative input; and the resistor group is extended with the transmit feedback resistor. Feeding the transmitted signal back advantageously reduces the electrical impedance of the output resistor or output resistors. This reduced impedance advantageously improves the SNR.

In an embodiment of the medical probe buffer, one of the at least one transmit ports is a ground port; the associated transmit buffer is a ground buffer; the input of the ground buffer is conductively coupled to a system ground; and the associated transmit resistor is a ground resistor. Defining a ground advantageously provides an absolute reference for improved identification, calculation and/or suppression of the noise sources thereby improving the SNR.

In an embodiment of the medical probe buffer, one of the at least one receive port is a signal input port; the associated receive buffer is a signal buffer; preferably the output of the signal buffer is provided for further processing; and the associated receive resistor is a signal resistor. Labelling the receive port as signal input port provides the advantage of improved identification, calculation and/or suppression of the noise sources thereby improving the SNR.

In an embodiment of the medical probe buffer, the at least one receive port is at least two receive ports; the at least one transmit port is one transmit port; and the transmit port is a ground port. This is an embodiment advantageously redistributing the resistors such that less resistors, specifically less resistors at the receiving wires or inputs, more specifically less noise sources, are present in the medical probe buffer for improving the SNR while still adhering to the requirement that the current through the human body does not exceed a safety threshold current when an electrical short-circuit fault occurs in at most one resistor in the resistor group.

In an embodiment of the medical probe buffer, the safety threshold current is a predefined safety threshold current. The predefined safety threshold current is typically depending on if the medical probe, whereof the medical probe buffer may be part, is used invasive in the human body or not. The predefined safety threshold current may depend on a medical standard such as the IEC 60601 standard, more specific the IEC 60601-1 standard, even more specific the most recent version being the IEC 60601-1 version 3.1 standard. The predefined safety threshold current together with the voltages applied in or provided to the medical probe buffer advantageously allow to calculate the resistance values of the different resistors in the resistor group.

In an embodiment of the medical probe buffer, the safety threshold current is in the range of 1 μA to 1 A, preferably 10 μA to 500 mA, more preferably 10 μA to 500 μA, more preferably 30 μA to 50 μA, more preferably 35 μA to 50 μA, most preferably below 50 μA. These safety threshold currents are typically preselected in a range such that the current is not lethal, not harmful or even not discomforting to the human being probed.

In an embodiment of the medical probe buffer, the measured physiological signal is a bioelectric signal. The bioelectrical signal may be a signal of the human body going to a muscle or generated in a muscle, such as a heart muscle contraction. The bioelectrical signal may be a signal of the human body of the brain or nerve system. The signal of the nerve system may be a signal from a receptor or one of the senses, such as the eye, ear, skin or the like. The signal of the nerve system may be an outgoing signal to e.g. a muscle, skin or organ. In an alternative embodiment, the physiological signal may be from a sensor attached or inserted in the human body, such as an attitude sensor for e.g., sensing the attitude of the human body for e.g. measuring movement of the human body.

In an embodiment of the medical probe buffer, the voltage supplied to the at least one receive port and the at least one transmit port is in the range of 100 mV to 100V, preferably 500 mV to 30V, more preferably 3V to 24V, most preferably around 3V, 5V, 12V or 24V. The voltages supplied to the different ports are typically preselected in a range such that the current resulting from the voltage over a resistance is not lethal, not harmful or even not discomforting to the human being probed.

In an embodiment of the medical probe buffer, each of the resistors has a resistance value in the range of 1KΩ to 10 MΩ, preferably 5KΩ to 1 MΩ, more preferably 10KΩ to 100KΩ, most preferably around 50KΩ. The resistors are typically preselected in a range such that in conjunction with the applied voltages and resulting current does not exceeds the safety threshold current even when an electrical short-circuit fault occurs. The resistors in the resistor group are preferably preselected with an equal value for all resistors in the group.

In an embodiment of the medical probe buffer, the medical probe comprises an IC package, wherein at least the buffers are arranged inside the IC package. The IC package advantageously provides an integration of at least part of the medical probe buffer reducing the number of components thereby simplifying the medical probe buffer.

In an embodiment of the medical probe buffer, the associated receive resistor is a distinct associated receive resistor. In a further preferred embodiment, each resistor in the resistor group is a distinct resistor. A distinct and/or discrete resistor advantageously separates resistors from each other for isolating an electrical short-circuit fault to one resistor such that an electrical short-circuit fault cannot bridge two or more resistors with one fault.

### BRIEF DESCRIPTION OF THE DRAWINGS

The invention will be apparent from and elucidated further with reference to the embodiments described by way of example in the following description and with reference to the accompanying drawings, in which:

FIG. 1 schematically shows a medical probe buffer according to the prior art;

FIG. 2 schematically shows a medical probe buffer according to the invention; and

FIG. 3 schematically shows a medical probe buffer according to the invention.

The FIGURES are purely diagrammatic and not drawn to scale. In the FIGURES, elements which correspond to elements already described may have the same reference numerals.

### LIST OF REFERENCE NUMERALS

| | |
|---|---|
| 10 | human |
| 11 | human body |
| 20 | prior art medical probe buffer |
| 100 | medical probe buffer |
| 105, 105′ | signal output |
| 110, 110′ | receive port |
| 120, 120′ | receive buffer |
| 121, 121′ | receive buffer input |
| 122, 122′ | receive buffer output |
| 125, 125′ | receive shield amplifier |
| 126, 126′ | receive shield amplifier positive input |
| 127, 127′ | receive shield amplifier negative input |
| 128, 128′ | receive shield amplifier output |
| 130, 130′ | receive shield port |
| 140, 140′ | receive shield buffer |
| 141, 141′ | receive shield buffer input |
| 142, 142′ | receive shield buffer output |
| 150 | transmit port |
| 160 | transmit buffer |
| 161 | transmit buffer input |
| 162 | transmit buffer output |
| 165 | transmit amplifier |
| 166 | transmit amplifier positive input |
| 167 | transmit amplifier negative input |
| 168 | transmit amplifier output |
| 200 | medical probe |
| 210, 210′, 210″ | cable |
| 220, 220′ | receive wire |
| 221, 221′ | receive wire shield |
| 250 | transmit wire |
| G | ground |
| R1a, R1a′ | receive resistors |
| R1b, R1b′, R1c, R1c′ | receive resistors, prior art |
| R2a, R2a′, R2b, R2b′ | receive shield resistor |
| R2c, R2c′ | receive resistors, prior art |
| R3a, R3a′ | receive shield feedback resistor |
| R4a, R4b | transmit resistors |
| R5a | transmit feedback resistor |
| Rg | resistor group |

### DETAILED DESCRIPTION OF THE FIGURES

The following FIGURES may detail different embodiments. Embodiments can be combined to reach an enhanced or improved technical effect. These combined embodiments may be mentioned explicitly throughout the text, may be hint upon in the text or may be implicit.

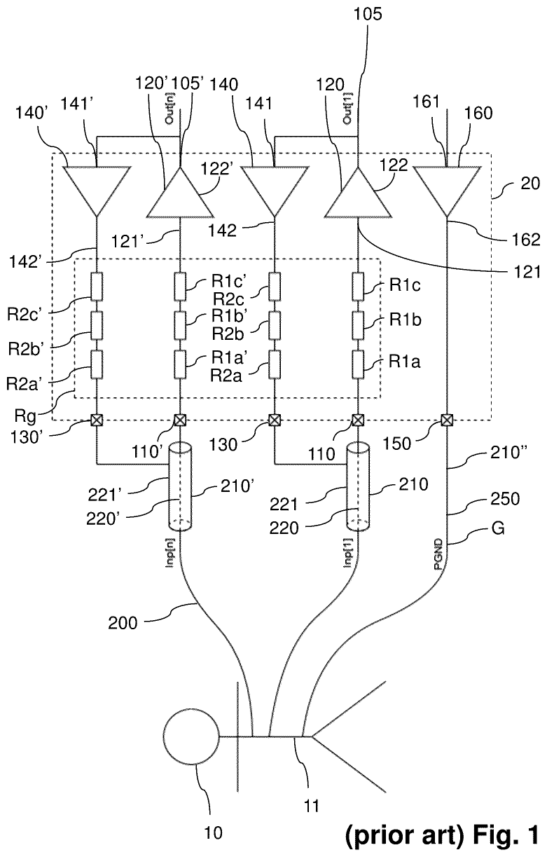

FIG. 1 schematically shows a medical probe buffer 20—with a dotted box-according to the prior art. The medical probe buffer is arranged for buffering a first measured physiological signal of a human body 11 of a human 10. The medical probe buffer according to the prior art comprises a first receive port 110, a first receive buffer 120, first receive resistors R1a, R1b, R1c, a second receive port 110′, a second receive buffer 120′, second receive resistors R1a′, R1b′, R1c′, a transmit port 150, and a transmit buffer 160.

The first receive buffer comprises a first receive buffer input 121 and a first receive buffer output 122. The first receive resistors conductively couple in series the first receive port and the first receive buffer input. The first receive buffer output provides the first signal 105.

The second receive buffer comprises a second receive buffer input 121′ and a second receive buffer output 122′. The second receive resistors conductively couple in series the second receive port and the second receive buffer input. The second receive buffer output provides the second signal 105′.

The medical probe buffer according to the prior art further comprises a first receive shield buffer 140, a second receive shield buffer 140′, first receive shield resistors R2a, R2b, R2c, second receive shield resistors R2a′, R2b′, R2c′.

The first receive shield buffer comprises a first receive shield buffer input 141 and a first receive shield buffer output 142. The first receive shield resistors conductively couple in series the first receive shield port and the first receive shield buffer output. The first receive shield buffer input is conductively coupled to the first receive buffer output.

The second receive shield buffer comprises a second receive shield buffer input 141′ and a second receive shield buffer output 142′. The second receive shield resistors conductively couple in series the second receive shield port and the second receive shield buffer output. The second receive shield buffer input is conductively coupled to the second receive buffer output.

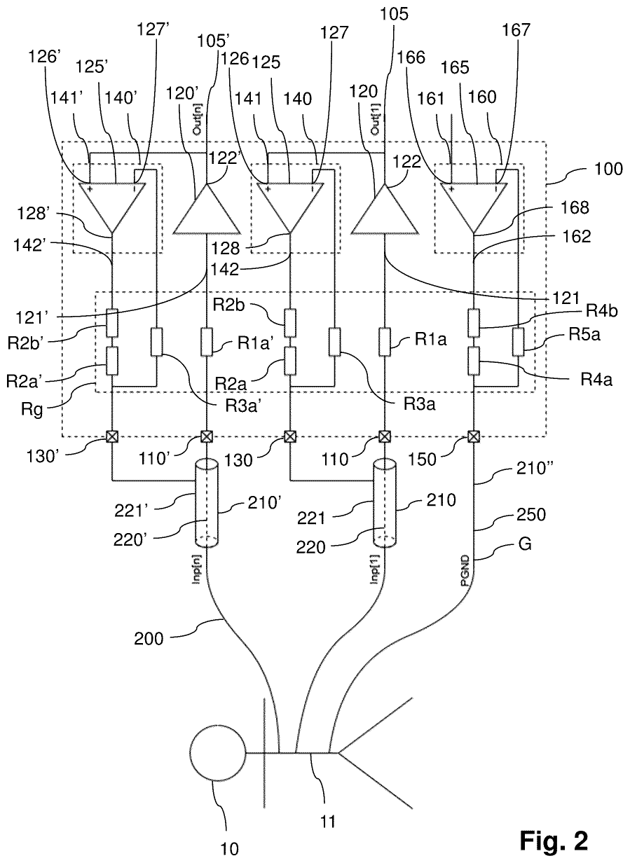

FIG. 2 schematically shows a medical probe buffer 100—with a dotted box-according to the invention. The medical probe buffer is arranged for buffering a first measured physiological signal of a human body 11 of a human 10. The medical probe buffer comprises a first receive port 110, a first receive buffer 120, a first receive resistor R1a, a transmit port 150, a transmit buffer 160, and a first transmit resistor R4a. The first receive buffer comprises a first receive buffer input 121 and a first receive buffer output 122.

The first receive port 110 is couplable to a first receive wire 220, which is arranged for conducting a first signal based on the first measured physiological signal of the human body. The first receive buffer is associated with the first receive port. The first receive buffer is arranged for buffering the signal from the first receive port. The first receive resistor is associated with the first receive port. The first receive resistor is conductively arranged between the first receive port and the first receive buffer input. The first receive buffer output is coupled to a first signal output 105 providing the buffered first measured physiological signal typically for further processing by a medical device or as part of a medical device.

The transmit buffer comprises a transmit buffer input 161 and a transmit buffer output 162. A transmit wire 250 is couplable to the transmit port. The transmit wire is arranged for conducting a signal to the human body, this signal may be labelled as the ground signal G or simply as ground G. The first transmit resistor is associated with the transmit port. The first transmit resistor is conductively arranged between the output of the transmit buffer and the transmit port.

A resistor group Rg is formed by the first receive resistor and the first transmit resistor. The resistance values of the resistors in the resistor group are preselected such that a current through the human body does not exceed a safety threshold current when an electrical short-circuit fault occurs in a resistor in the resistor group.

The resistor group is part of the MOP of the medical probe buffer. Determining the resistance values of the resistors in the resistor group is therefore done following a worst-case scenario. First the conductive path in case of the first receive buffer is thus at least formed by the transmit buffer (160), transmit resistor (R4a), transmit port (150), transmit wire (250), human body (11), receive wire (220), receive port (110), receive resistor (R1a), and receive buffer (120).

When preselecting the resistance values of the transmit and receive resistors for use as means of patient protection (MOP), the following assumptions are made:
the output resistance of the transmit buffer is to be assumed to have zero resistance;
the transmit port is to be assumed to have zero resistance;
the transmit wire is to be assumed to have zero resistance;
the human body is to be assumed to have zero resistance;
the receive wire is to be assumed to have zero resistance;
the receive port is to be assumed to have zero resistance; and
the input of the receive buffer is to be assumed to have zero resistance.

Under these conditions, the transmit resistor should have a resistance value high enough to limit the current to below the safety threshold current when an electrical short-circuit occurs in the receive resistor. And vice versa, under these conditions, the receive resistor should have a resistance value high enough to limit the current to below the safety threshold current when an electrical short-circuit occurs in the transmit resistor.

Based on the procedure above, a minimum resistance value for the transmit and receive resistors can be calculated. Further, any resistance value higher than the minimum resistance value will suffice the limitation of this feature. Hence, a range of resistance values suffices the limitation of this feature. A resistance value for the transmit and receive resistors from the range of resistance values is preselected such that a current through the human body does not exceed a safety threshold current when an electrical short-circuit fault occurs in either the transmit or the receive resistors.

The medical probe buffer may comprise a second receive port 110′. The second receive port is couplable to a second receive wire 220′, which is arranged for conducting a second signal based on the second measured physiological signal of the human body. The medical probe buffer may comprise a second receive buffer 120′. The second receive buffer comprises a second receive buffer input 121′ and a second receive buffer output 122′. The second receive buffer is associated with the second receive port. The second receive buffer is arranged for buffering the signal from the second receive port. The medical probe buffer may comprise a second receive resistor R1a′. The second receive resistor is associated with the second receive port. The second receive resistor is conductively arranged between the second receive port and the second receive buffer input. The second receive buffer output is coupled to a second signal output 105′ providing the buffered second measured physiological signal typically for further processing by a medical device or as part of a medical device.

The medical probe buffer may comprise second transmit resistor R4b. The second transmit resistor is conductively arranged between the output of the transmit buffer and the transmit port. The first and second transmit resistors are typically conductively coupled in series. The first and second transmit resistors are typically discrete resistors. The second transmit resistor is added to the resistor group. The requirement that the resistance values of the resistors in the resistor group are preselected such that a current through the human body does not exceed a safety threshold current when an electrical short-circuit fault occurs in a resistor in the resistor group remains the same. The effect of adding the second transmit resistor to the resistor group is that the resistor value per resistor may be lower. Furthermore, the total resistor value of the resistors may be lower, because if one resistor fails due to a short-circuit the resistor values of the other resistors in the resistor group are to be added to come to a value complying with the requirement that a current through the human body does not exceed a safety threshold current. This lower total resistor value causes an improved SNR of the signal, specifically the received signal. Further, the arrangement and/or the placement in the electrical circuit provides the effect that the noise of the resistors, more specifically of the transmit resistors, have a reduced influence on the signal provided by at least the first receive buffer, preferably when present also the receive buffers.

The medical probe buffer may comprise a receive shield port 130, 130′ associated with a receive port 110, 110′. The receive shield port is couplable to a shield 221, 221′. The shield is arranged for electrically shielding the receive wire 220, 220′ coupled to the associated receive port. The medical probe buffer may comprise a shield buffer 140, 140′. Each of the shield buffers is associated with a receive shield port. The shield buffer comprises a shield buffer input 141, 141′ and a shield buffer output 142, 142′. The medical probe buffer may comprise a receive shield resistor R2a, R2a′, R2b, R2b′. Each of the receive shield resistors is associated with a receive shield port. The receive shield resistors R2a, R2b are conductively arranged in series between the receive shield buffer output 142 and the receive shield port 130. The receive shield resistors R2a′, R2b′ are conductively arranged in series between the receive shield buffer output 142′ and the receive shield port 130′. In an alternative embodiment, only one receive shield resistor R2a, R2a′ is present per associated receive shield port and/or receive port. In an alternative embodiment, a plurality of receive shield resistors are present per associated receive shield port and/or receive port. The resistor group is extended with the receive shield resistors. The requirements that the resistance values of the resistors in the resistor group are preselected such that a current through the human body does not exceed a safety threshold current when an electrical short-circuit fault occurs in a resistor in the resistor group remains.

The at least one receive shield buffer 140, 140′ may comprise a receive shield amplifier 125, 125′. The receive shield amplifier comprises a receive shield amplifier output 128, 128′, a receive shield amplifier negative input 127, 127′, and a receive shield amplifier positive input 126, 126′. The receive shield amplifier output is conductively coupled to the output of the receive shield buffer. The receive shield amplifier positive input is conductively coupled to the receive shield buffer input. The medical probe buffer may comprise a receive shield feedback resistor R3a, R3a′ conductively coupling the associated receive shield port and the receive shield amplifier negative input. The resistor group is extended with the receive shield resistors. The requirements that the resistance values of the resistors in the resistor group are preselected such that a current through the human body does not exceed a safety threshold current when an electrical short-circuit fault occurs in a resistor in the resistor group remains.

The at least one transmit buffer may comprise a transmit amplifier 165. The transmit amplifier comprises a transmit amplifier output 168, a transmit amplifier negative input 167, and a transmit amplifier positive input 166. The transmit amplifier output is conductively coupled to the output of the transmit buffer. The transmit amplifier positive input is conductively coupled to the transmit buffer input. The medical probe buffer comprises a transmit feedback resistor R5a conductively coupling the associated transmit port and the transmit amplifier negative input. The resistor group is extended with the transmit feedback resistor. The requirement that the resistance values of the resistors in the resistor group are preselected such that a current through the human body does not exceed a safety threshold current when an electrical short-circuit fault occurs in a resistor in the resistor group remains.

A medical probe 200 may comprise, a medical probe buffer 100, and at least one probe cable 210, 210′, 210″. The at least one cable may be and/or comprise a first receive wire 220, a second receive wire 220′, a further receive wire, and/or a transmit wire 250. The probe cable may comprise a wire arranged for conducting the signal and is couplable to a port of the medical probe buffer. The cable may comprise a shield associated with a wire for shielding the wire from electrical magnetic interference. The shield is couplable to a shield port associated with the port whereto the wire is couplable and that is associated with the shield.

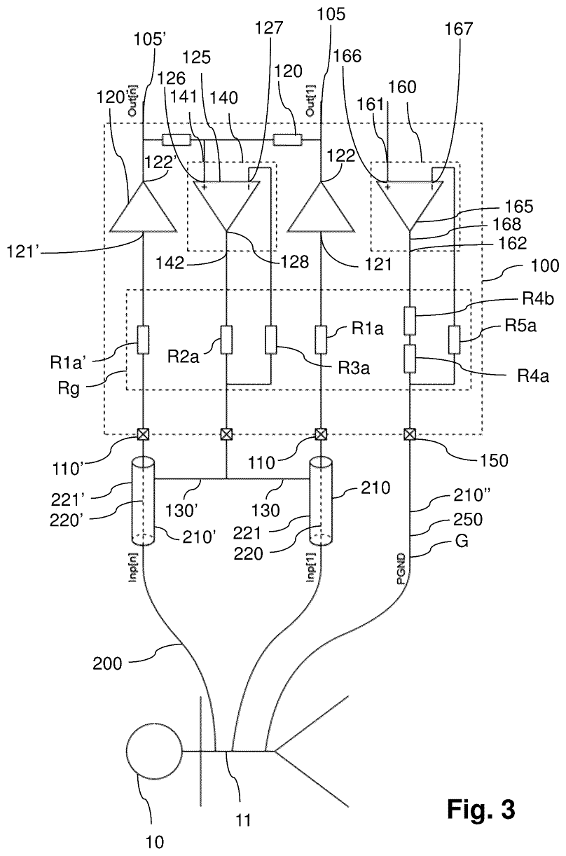

FIG. 3 schematically shows a medical probe buffer 100—with a dotted box-according to the invention. The medical probe buffer in FIG. 3 shows the medical probe buffer in FIG. 2 extended with stability functionality.

The medical probe buffer in FIG. 3 comprises only one receive shield buffer 140 providing a feedback shield signal for the shields of the first receive wire 220 and the second receive wire 220′. The receive buffer comprises a receive shield buffer input 141, and a receive shield buffer output 142.

The medical probe buffer may further comprise a signal averaging circuit. The signal averaging circuit comprise two connections, respectively coupled to the first and second received signals. The two connections are conductively coupled with two series resistors. The receive shield buffer input 141 is conductively coupled to the electrical point coupling the two series resistors. This provides the advantage of an averaged and typically more balanced input signal to the receive shield buffer. The receive shield buffer output is via a receive shield resistor R2a conductively coupled to a first and a second receive shield 221, 221′ respectively shielding the first and the second receive wire. Electrical interference is typically interfering both the receive wires about the same. The current embodiment provides the advantage of reducing the number of components in the medical probe buffer thereby increasing the mean time between failure. The current embodiment provides the advantage of averaging the electrical interference of two received signals for damping or reducing the received shield signal thereby advantageously limiting extremes in the receive shield signal preventing or limiting this receive shield signal from introducing an interference in itself.

The resistors in the resistor group are preferably preselected with an equal value for all resistors in the group. This provides a low or even lowest overall or total resistance to the received signal. Lowering the total resistance lowers the generated noise, thus improves the SNR for the received signal.

An alternative embodiment may be a medical probe buffer for buffering a measured physiological signal of a human body, comprising: at least one receive port couplable to a receive wire, which is arranged for conducting a signal based on a physiological signal of the human body; each receive port having an associated receive buffer arranged for buffering the signal from the respective receive port, wherein each receive buffer comprises an input, and an output; each receive port having an associated conductive receiving path providing conduction between the associated receive port and the associated receive buffer input, wherein the conductive receiving path comprises at least one receive resistor; at least one transmit port couplable to a transmit wire, which is arranged for conducting a signal to the human body; each transmit port having an associated transmit buffer arranged for buffering the signal provided to the respective transmit port, wherein each transmit buffer comprises an input, and an output; and each transmit port having an associated conductive transmitting path providing conduction between the associated transmit port and the associated transmit buffer output, wherein the conductive transmitting path comprises at least one transmit resistor; wherein a resistor group is formed by the resistors in the conductive receiving path and the resistors in the conductive transmitting path; and wherein the resistance values of the resistors in the resistor group are preselected such that a current through the human body does not exceed a safety threshold current when an electrical short-circuit fault occurs in a resistor in the resistor group. The arrangement of the resistors in this embodiment or in other embodiments, more specifically the positioning of the resistors, provide for that an electrical short-circuit in one of the electrical components, such as in one of the buffers or resistors, will not cause a current through the human body to exceed a safety threshold current. This alternative embodiment may provide the same or comparable technical effects or advantages as described. The alternative embodiment may be combined with features from other embodiments or features of embodiments for obtaining the same or comparable technical effects or advantages as described.

Examples, embodiments or optional features, whether indicated as non-limiting or not, are not to be understood as limiting the invention as claimed. It should be noted that the FIGURES are purely diagrammatic and not drawn to scale. In the FIGURES, elements which correspond to elements already described may have the same reference numerals.

The term “substantially” herein, such as in “substantially all emission” or in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”.

The term “functionally” will be understood by, and be clear to, a person skilled in the art. The term “substantially” as well as “functionally” may also include embodiments with “entirely”, “completely”, “all”, etc. Hence, in embodiments the adjective functionally may also be removed. When used, for instance in “functionally parallel”, a skilled person will understand that the adjective “functionally” includes the term substantially as explained above. Functionally in particular is to be understood to include a configuration of features that allows these features to function as if the adjective “functionally” was not present. The term “functionally” is intended to cover variations in the feature to which it refers, and which variations are such that in the functional use of the feature, possibly in combination with other features it relates to in the invention, that combination of features is able to operate or function. For instance, if an antenna is functionally coupled or functionally connected to a communication device, received electromagnetic signals that are receives by the antenna can be used by the communication device. The word “functionally” as for instance used in “functionally parallel” is used to cover exactly parallel, but also the embodiments that are covered by the word “substantially” explained above. For instance, “functionally parallel” relates to embodiments that in operation function as if the parts are for instance parallel. This covers embodiments for which it is clear to a skilled person that it operates within its intended field of use as if it were parallel.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices or apparatus herein are amongst others described during operation. As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device or apparatus claims enumerating several means, several of these means may be embodied by one and the same item of hardware. The mere fact that certain measures are recited in mutually different dependent claims does not indicate that a combination of these measures cannot be used to advantage.

The invention further applies to an apparatus or device comprising one or more of the characterising features described in the description and/or shown in the attached drawings. The invention further pertains to a method or process comprising one or more of the characterising features described in the description and/or shown in the attached drawings.

The various aspects discussed in this patent can be combined in order to provide additional advantages. Furthermore, some of the features can form the basis for one or more divisional applications.

1. A medical probe buffer for buffering a measured physiological signal of a human body, comprising:
at least one receive port couplable to a receive wire, which is arranged for conducting a signal based on a physiological signal of the human body;
each receive port having an associated receive buffer arranged for buffering the signal from the respective receive port, wherein each receive buffer comprises an input, and an output;
each receive port having an associated receive resistor conductively arranged between the respective receive port and the input of the respective receive buffer;
at least one transmit port couplable to a transmit wire, which is arranged for conducting a signal to the human body;
each transmit port having an associated transmit buffer arranged for buffering the signal provided to the respective transmit port, wherein each transmit buffer comprises an input, and an output; and
each transmit port having a number of associated transmit resistors conductively arranged in series between the output of the transmit buffer and the transmit port;
wherein a resistor group is formed by the receive resistor and the number of transmit resistors; and
wherein the resistance values of the resistors in the resistor group are preselected such that a current through the human body does not exceed a safety threshold current when an electrical short-circuit fault occurs in a resistor in the resistor group.

2. The medical probe buffer according to claim 1, wherein the number of receive ports is at least two.

3. The medical probe buffer according to claim 1,
wherein the medical probe buffer comprises means of protection;
wherein the means of protection comprises the resistor group; and
wherein the resistance values of the resistors in the resistor group are preselected such that a current through the human body does not exceed a safety threshold current when next to the electrical short-circuit fault occurring in the resistor in the resistor group also an electrical short-circuit fault occurs in the medical probe buffer outside the means of protection.

4. The medical probe buffer according to claim 1, wherein the number of transmit resistors connected in series is two.

5. The medical probe buffer according to claim 1, comprising:
a receive shield port associated with one of the at least one receive ports and couplable to a shield arranged for electrically shielding the receive wire coupled to the associated receive port;
each receive shield port having an associated shield buffer comprising an input and an output; and
each receive shield port having an associated receive shield resistor conductively arranged between the output of the receive shield buffer and the receive shield port;
wherein the resistor group is extended with the receive shield resistor.

6. The medical probe buffer according to claim 5,
wherein the receive shield resistor comprises a number of receive shield resistors connected in series; and
wherein the receive shield resistor in the resistor group is replaced with the number of receive shield resistors.

7. The medical probe buffer according to claim 6, wherein the number of receive shield resistors in series is two.

8. The medical probe buffer according to claim 5,
wherein the at least one receive shield buffer comprises a receive shield amplifier comprising a receive shield amplifier output, a receive shield amplifier negative input, and a receive shield amplifier positive input;
wherein the receive shield amplifier output is conductively coupled to the output of the receive shield buffer;
wherein the receive shield amplifier positive input is conductively coupled to the receive shield buffer input;
wherein the medical probe buffer comprises a receive shield feedback resistor conductively coupling the associated receive shield port and the receive shield amplifier negative input; and
wherein the resistor group is extended with the receive shield feedback resistor.

9. The medical probe buffer according to claim 1,
wherein the at least one transmit buffer comprises a transmit amplifier comprising a transmit amplifier output, a transmit amplifier negative input, and a transmit amplifier positive input;
wherein the transmit amplifier output is conductively coupled to the output of the transmit buffer;
wherein the transmit amplifier positive input is conductively coupled to the transmit buffer input;
wherein the medical probe buffer comprises a transmit feedback resistor conductively coupling the associated transmit port and the transmit amplifier negative input; and
wherein the resistor group is extended with the transmit feedback resistor.

10. The medical probe buffer according to claim 1,
wherein one of the at least one transmit port is a ground port;
wherein the associated transmit buffer is a ground buffer;
wherein the input of the ground buffer is conductively coupled to an external ground; and
wherein the associated transmit resistor is a ground resistor.

11. The medical probe buffer according to claim 1,
wherein one of the at least one receive port is a signal input port;
wherein the associated receive buffer is a signal buffer; and
wherein the associated receive resistor is a signal resistor.

12. The medical probe buffer according to claim 1,
wherein the at least one receive port is at least two receive ports;
wherein the at least one transmit port is one transmit port; and
wherein the transmit port is a ground port.

13. The medical probe buffer according to claim 1, wherein the safety threshold current is a predefined safety threshold current.

14. The medical probe buffer according to claim 1, wherein the safety threshold current is in the range of 1 μA to 1 A.

15. The medical probe buffer according to claim 1, wherein the measured physiological signal is a bioelectric signal.

16. The medical probe buffer according to claim 1, wherein the voltage supplied to the at least one receive port and the at least one transmit port is in the range of 100 mV to 100V, and the resistors has a resistance value in the range of 1KΩ to 10 MΩ.

17. (canceled)

18. The medical probe buffer according claim 1, comprising an IC package, wherein at least the buffers are arranged inside the IC package.

19. The medical probe buffer according to claim 1, wherein the associated receive resistor is a distinct associated receive resistor.

20. A medical probe comprising:
a medical probe buffer according to for buffering a measured physiological signal of a human body, comprising:
at least one receive port couplable to a receive wire, which is arranged for conducting a signal based on a physiological signal of the human body;
each receive port having an associated receive buffer arranged for buffering the signal from the respective receive port, wherein each receive buffer comprises an input, and an output;
each receive port having an associated receive resistor conductively arranged between the respective receive port and the input of the respective receive buffer;
at least one transmit port couplable to a transmit wire, which is arranged for conducting a signal to the human body;
each transmit port having an associated transmit buffer arranged for buffering the signal provided to the respective transmit port, wherein each transmit buffer comprises an input, and an output; and
each transmit port having a number of associated transmit resistors conductively arranged in series between the output of the transmit buffer and the transmit port;
wherein a resistor group is formed by the receive resistor and the number of transmit resistors; and
wherein the resistance values of the resistors in the resistor group are preselected such that a current through the human body does not exceed a safety threshold current when an electrical short-circuit fault occurs in a resistor in the resistor group; and
at least one probe cable comprising:
a receive wire couplable to the at least one receive port; and
a transmit wire couplable to the at least one transmit port.

21. A medical device for measuring a physiological signal of a human body, comprising:
a medical probe arranged for measuring the physiological signal;
a medical probe buffer arranged for receiving the measured physiological signal from the probe, the probe comprising:
at least one receive port couplable to a receive wire, which is arranged for conducting a signal based on a physiological signal of the human body;
each receive port having an associated receive buffer arranged for buffering the signal from the respective receive port, wherein each receive buffer comprises an input, and an output;
each receive port having an associated receive resistor conductively arranged between the respective receive port and the input of the respective receive buffer;
at least one transmit port couplable to a transmit wire, which is arranged for conducting a signal to the human body;
each transmit port having an associated transmit buffer arranged for buffering the signal provided to the respective transmit port, wherein each transmit buffer comprises an input, and an output; and
each transmit port having a number of associated transmit resistors conductively arranged in series between the output of the transmit buffer and the transmit port;
wherein a resistor group is formed by the receive resistor and the number of transmit resistors; and
wherein the resistance values of the resistors in the resistor group are preselected such that a current through the human body does not exceed a safety threshold current when an electrical short-circuit fault occurs in a resistor in the resistor group; and
a processor for processing the buffered physiological signal from the medical probe buffer.
